(19) United States
(12) Patent Application Publication
(10) Pub. No.: US 2024/0197711 A1
(43) Pub. Date: Jun. 20, 2024

(54) Combination Therapy for Cancer Treatment

(71) Applicant: THE REGENTS OF THE UNIVERSITY OF MICHIGAN, Ann Arbor, MI (US)

(72) Inventors: Mukesh K. Nyati, Superior Township, MI (US); Ranjit Kumar Mehta, Ann Arbor, MI (US)

(21) Appl. No.: 18/284,892

(22) PCT Filed: Apr. 1, 2022
(86) PCT No.: PCT/US22/22990

§ 371 Date: Sep. 29, 2023

Related U.S. Application Data
(60) Provisional application No. 63/170,038, filed on Apr. 2, 2021.

(51) Int. Cl. A61K 31/4709; A61P 35/00

(52) U.S. Cl. CPC A61K 31/4709; A61P 35/00

(57) ABSTRACT

Provided herein are methods of treating a cancer comprising administering an EGFR degrader to a patient suffering therefrom and subjecting the patient to radiation. The cancer can express mutant, overexpressed or overly activated EGFR, mutant KRAS, or mutant BRAF.

### STATEMENT OF GOVERNMENT INTEREST

This invention was made with government support under 1 R01 CA248310-01, awarded by the National Institutes of Health. The government has certain rights in the invention.

### BACKGROUND

Three common oncogenes in solid tumors, EGFR, KRAS and BRAF are seen to be mutated or overexpressed in over half of all solid tumors, but these mutations/overexpression's are usually mutually exclusive, and most tumors only show defects in one of the three. There are several inhibitors of EGFR on the market, but for small molecules, their efficacy is entirely limited to a subset of non-small cell lung cancers (NSCLC) which express mutant EGFR, and for antibodies a similar percentage of colorectal cancers (CRC), and head and neck squamous cell carcinomas (HNSCC) in combination with radiation. It has been shown in repeated clinical trials that patients with either KRAS or BRAF mutations do not respond to EGFR inhibition, regardless of EGFR status. As both of these oncogenes are downstream of EGFR in very important proliferative and survival signaling pathways, the lack of efficacy for EGFR inhibition can be quite readily explained by its signaling being rendered redundant by the downstream mutations.

EGFR is a Receptor Tyrosine Kinase (RTK), and it provides proliferative, survival, metabolic and motility signals into cells principally by binding its cognate receptors such as EGF, HB-EGF, AMPR, EPG and TGFα. Ligand binding leads to activation of a tyrosine kinase, which leads to specific phosphorylation of tyrosine hydroxyls on a large number of substrate proteins including EGFR itself. This activity recruits and activates a whole series of signaling cascades (mainly through serine/threonine phosphorylation) at the cell surface, and these cascades signal into the nucleus, leading to gene expression changes which facilitate proliferation, and other activities described earlier. In cells, and in animal tumor models, EGFR tyrosine kinase inhibitors (TKI) lead to very profound and general anti-tumor activity in tumors which overexpress EGFR or have mutant EGFR. However, they are of much lesser effect in models driven by mt-KRAS and mt-BRAF, as expected. Similarly, antibodies which prevent EGFR TK activation show very good activity in preclinical models where EGFR is the driving oncogene.

However, once in the clinic, EGFR inhibitors/antibodies proved to have a much less broad spectrum of activity against EGFR-driven tumors than expected from pre-clinical models. The TKIs only work against mt-EGFR, which is only common in NSCLCs (˜15% occurrence), and antibodies only work against a subset of wt-EGFR expressing CRCs, and some HNSCCs. Overall, the majority of apparently EGFR-driven tumors respond to neither therapeutic modality in the clinic, and as expected there is no clinical activity seen in mt-KRAS and mt-BRAF driven solid tumors. Currently, there are no approved KRAS-directed therapies, although agents targeted at a minor KRAS mutant oncogene are in clinical trials. Just as there are clinically approved kinase inhibitors for EGFR, there are also for mt-BRAF. However, in all of these cases there are dose limiting toxicities, and even the most dramatic responses to the drugs are transient, with virtually no patients getting more than two years of progression free survival, and most considerably less. Thus there is still a great unmet medical need for all of these tumor types.

### SUMMARY

Provided herein are methods of treating cancer in a patient suffering therefrom comprising administering to the patient an EGFR degrader, and subjecting the patient to radiation to treat the cancer.

In various cases, the EGFR degrader degrades wild type EGFR. In various cases, the EGFR degrader degrades mutant EGFR. In some cases, the EGFR degrader is a compound (or pharmaceutically acceptable salt thereof), an antibody, a protein, a peptide, a PROTAC (proteolysis targeting chimera), a virus, an antibody-drug conjugate, an aptamer, a peptidomimetic agent, or an oligonucleotide. In some cases, the compound has a structure of formula (I):

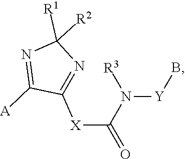

or in some cases, a structure of Compound A:

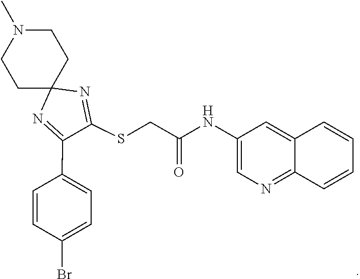 [SMILES: CN1CCC2(CC1)N=C(SCC(=O)Nc1cnc3ccccc3c1)C(c1ccc(Br)cc1)=N2]

In various cases, the cancer is an EGFR, KRAS, or BRAF mutated cancer. In some cases, the cancer is a solid tumor. In some cases, the cancer is pancreatic cancer, colorectal cancer, head and neck cancer, or lung cancer.

### BRIEF DESCRIPTION OF FIGURES

FIG. 1 shows tumor volume of mice having (A) PC9 (mutant EGFR tumor), (B) RKO (mutant BRAF tumor), and (C) UMSCC74B (mutant KRAS tumor) over time after treatment with Compound A (shown as DPI-503), radiotherapy (designated RT), and combination of Compound A and radiotherapy (designated DPI-503+RT), compared to control.

### DETAILED DESCRIPTION

Provided herein are methods of treating cancer in a patient by administering a chemotherapeutic and administering radiation to the patient. Surprisingly, it has been found that combination of a chemotherapeutic, whose specific mode of action is to degrade overactivated EGFR seen in many solid tumors, regardless of mutational status, with radiation has an unexpectedly improved effect on treating the cancer compared to the chemotherapeutic or radiation alone, if that cancer is driven by mutations in EGFR, KRAS, or BRAF oncogenes.

Overactivation-Driven Degraders of EGF Receptor (ODDER) compounds interact with EGFR in a way which does not appear to affect its kinase activity, so they are not tyrosine kinase inhibitors (TKIs). However, binding of ODDERs to EGFR prevents activated EGFR from forming the stable molecular complexes required to protect them from being rapidly internalized and degraded by the normal cellular protein degradation mechanisms, which is exactly what these molecules were designed to do. After EGFR has been activated, in normal tissues by binding of a cognate ligand the kinase domain changes its conformation, from an inactive conformation, which cannot bind ATP or substrate, to an active one, which allows both ATP and substrate to bind to the enzyme, which then catalyzes the transfer of the γ-phosphate of ATP to the phenolic hydroxy group of a tyrosine side chain. The kinase catalytic domain then unbinds both the product, a tyrosyl phosphate monoester, and the byproduct ADP, and can then bind further ATP and substrate as the catalytic cycle continues. There appear to be two major uses for tyrosine phosphorylation in a substrate molecule. The first is that conformational changes in the substrate protein are often driven by the conversion of a neutral, somewhat hydrophobic, tyrosine side chain into a highly hydrophilic phosphotyrosine, with two negative charges at physiological pH. The second is that there are binding sites on many proteins for phosphotyrosine residues, which induce new protein-protein interactions for the substrate protein, frequently changing its cellular localization and/or allowing it to assemble into new multiprotein complexes, both of which can lead the substrate protein to show very different activities after phosphorylation. Significantly, several of the most important substrate tyrosines for EGFR are on its own C-terminal domain (CTD), which is in the cell's cytoplasm. This recruits the intermediate proteins which lead to the activation of the before mentioned signaling cascades. However, some of these phosphorylations also lead to recruitment of proteins which cause the receptor to be rapidly internalized into the cell via several different pathways, and ends up in intracellular vesicles called endosomes. Endosome traffic throughout the cell, sometimes changing their properties as they go, and ultimately sort trafficked proteins like EGFR into endosomes which return the receptors to the cell surface, where they can continue signaling, or to lysosomes, where they are degraded and become non-functional. This is a complex process, but when ODDERs are present, they seem to have minor effects on unactivated or weakly activated EGFR, but lead to quite rapid and efficient diversion of the highly activated EGFR typically seen in tumors into the degradation pathway, which leads to an overall loss of EGFR protein in the treated cells over a 6-24 hour period depending on the cell type. Interestingly, this degradation can be blocked by use of classical EGFR inhibitor co-treatment, showing the importance of kinase activation to this mechanism of action. As a no longer present protein cannot act as a TK, one effect of this protein degradation is to act functionally as an EGFR kinase TKI, and as expected these compounds have potent activity in certain EGFR-driven tumor xenograft models.

Although EGFR has a vital role in early developmental processes, such that EGFR−/− mice only survive a few days after birth, a severe hypomorph (94% loss of enzyme activity), the waved-2 mouse has a remarkably mild phenotype, with certain hair and skin problems, and premature opening of the eyes, but these animals are very susceptible to GI tract injury, which they have difficulty healing. Wound-healing studies carried out on immundeficient mice grafted with either wt-EGFR containing skin, or EGFR−/− skin also demonstrate that the latter is highly defective in wound healing, suggesting that this is the major role for EGFR after the neonatal stage. Wound healing attributes include proliferation of cells to fill the wound, angiogenesis to get new blood vessels to support the new tissues, resistance to apoptosis, as wounds often allow for foreign toxins (including infection) to enter the injury site, and greatly increased nutrient inflow into cells to support the rapid anabolic metabolism required to produce new tissue as fast as possible. EGFR expression and activation leads to all of these sequelae, which of course are all ideal attributes for an oncogene.

Several membrane transporters are induced when EGFR kinase is activated via the downstream kinase cascades, and can be depleted by kinase inhibition. However, in the dysregulated environment of tumor cells, most of these pathways tend to be quite rapidly re-activated by alternative pathways, so the kinase-dependent effects on nutrient transporters are often transient. It has been shown that EGFR has kinase independent functions (KIFs), largely due to its ability to function not only as a scaffolding protein both for the kinase cascade signaling complexes, but also for several membrane bound proteins which have no direct function in the kinase signaling cascades. Several of these EGFR scaffold-dependent proteins are also membrane nutrient transporters, for example transporters for glucose and cysteine, which help to support increases metabolic growth in cells, allowing them to anabolize more efficiently, which in turn allows for enhanced proliferation. It has been reported that loss of EGFR via siRNA in cells leads to loss of several such transporters from the cell surface, and it has been shown that treatment of cells with ODDERs leads to loss of some of these transporters from the cell surface, presumably because a complex with the EGFR protein is required for them to exist stably at the cell membrane.

Additionally, surprising data from an NC160 Tumor Cell panel on Compound A (an ODDER compound) showed considerable anti-proliferative activity in several tumors driven by mt-KRAS or mt-BRAF, many of which are well established to be completely unaffected by EGFR TKIs or antibodies. ODDER compounds have also been shown to have anti-cancer activity in vivo in mt-KRAS and mt-BRAF tumors.

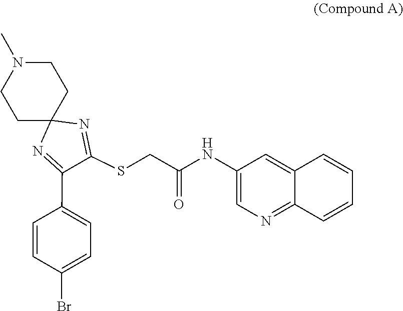 [SMILES: CN1CCC2(CC1)N=C(SCC(=O)Nc1cnc3ccccc3c1)C(c1ccc(Br)cc1)=N2]

Examination of the affected cell lines shows that they all coexpress wild-type (wt)—EGFR along with the KRAS or BRAF oncogene, and that the ODDER compound treatment leads to depletion of EGFR from the tumor cells. The successful ablation of EGFR from the cells occurs because, although they are not the driving oncogene in these tumors, both mt-KRAS and mt-BRAF downstream effects lead to strong activation of EGFR, and frequently a non-genomically driven overexpression, making the highly phosphorylated protein susceptible to ODDER-induced degradation.

It was reasoned that the activity seen in these susceptible tumor lines was largely due to EGFR KIFs. Tumors cells are always metabolically stressed, and in actual tumors poor circulation makes the stress even more severe on the tumor interior, which is usually hypoxic once the tumor has growth to more than 100 mm³. Experiments showed that a specific ODDER, Compound A, led to loss of EGFR from the cell surface, and that two important transporters known to require EGFR chaperoning, SGLT1, a sodium-dependent glucose transporter which imports glucose, and xCT, the cysteine-glutamate co-transporter which imports cysteine, were ablated along with EGFR. As tumor cells try to run in oxidative glycolysis, they have a much larger glucose requirement than normal cells, and any hindrance of glucose import would be expected to stress cells, pushing them away from aerobic glycolysis, and towards oxidative phosphorylation to produce ATP, which in turn would almost certainly increase production of reactive oxygen intermediates (ROI) in these already abnormally metabolizing cells. The most common ways of disposing of ROI are via conjugation with glutathione, or reaction with various reducing enzymes, most of which rely on cysteine thiols for their RedOx ability. Both of these mechanisms require stressed cells to be able to import large amounts of cysteine, much of which is produced by the liver and put into circulation, as the high demand totally overwhelms endogenous cysteine biosynthesis (which requires methionine as its source of sulfur). Thus, a loss of EGFR protein should increase oxidative stress in any tumor cell, both from the forced change in metabolism and from the dearth of intracellular cysteine, and it was reasoned that this could lead to mt-KRAS and mt-BRAF driven tumors being much more susceptible to EGFR ablation than to simple EGFR inhibition. Indeed, as EGFR inhibition usually leads to increased cell surface EGFR by blocking the kinase activity needed to trigger endocytosis and degradation, as well as perhaps slowing overall metabolism somewhat, simple EGFR inhibition in these cell types might decrease oxidative stress.

Signs of oxidative stress in cells were investigated after addition of increasing amounts of Compound A with hydrogen peroxide levels being chosen as a marker of increased oxidative stress. At six hours post treatment, there was a dose-dependent increase in H₂O₂ levels seen, with the top dose tested leading to 2-4-fold increases in cellular levels, showing that indeed EGFR ablation increases cellular oxidative stress in cells driven by mt-KRAS, mt-BRAF, and mt-EGFR. Consideration was given to ways of exploiting oxidative stress in these tumor types. Oxidative stress both contributes towards causing DNA damage, and makes DNA damage more difficult to repair. So a combination of an ODDER (e.g., Compound A) with a DNA damaging agent, such as radiation, may result in useful therapeutic regimen. Compound A and radiation was examined in vivo xenograft experiments in mt-EGFR, mt-KRAS, and mt-BRAF driven tumors. What was found was that radiation anti-tumor effects are strongly increased in the presence of Compound A in all three experiments. Thus, it is believed that ODDER compounds, such as Compound A, can be used in combination with radiation to treat localized tumors which express either mutant KRAS or mutant BRAF. It is also noted that, due to its unique mode of action, Compound A has much less on target toxicity than EGFR inhibitors, and it is expected that it can be used in the clinic at a low toxicity dose, in combination with standard radiotherapy regimens. Other ODDER compounds are also expected to have similar lower on target toxicity.

### EGFR Degraders

The EGFR degrader used in the methods disclosed herein can be any entity that degrades mutant EGFR such as a compound (or pharmaceutically acceptable salt thereof), an antibody, a protein, a peptide, a PROTAC (proteolysis targeting chimera), a virus, an antibody-drug conjugate, an aptamer, a peptidomimetic agent, or an oligonucleotide.

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A., 90; 6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

The term “antibody drug conjugate” as used herein refers to the linkage of an antibody or an antigen binding fragment thereof with another agent, such as a small molecule, peptide, an imaging probe, or the like. The linkage can be covalent bonds, or non-covalent interactions such as through electrostatic forces. Various linkers, known in the art, can be employed in order to form the antibody drug conjugate. Additionally, the antibody drug conjugate can be provided in the form of a fusion protein that may be expressed from a polynucleotide encoding the antibody drug conjugate.

As used herein, the terms “polypeptide” and “protein” are used interchangeably to refer to a polymer of amino acid residues, and are not limited to a minimum length. The terms include post-expression modifications of the polypeptide, for example, glycosylation, acetylation, phosphorylation, and the like. Furthermore, a “polypeptide” may refer to a protein which includes modifications, such as deletions, additions, and substitutions (generally conservative in nature), to the native sequence, as long as the protein maintains the desired activity. These modifications may be deliberate or may be accidental. A peptide refers to a fragment of a protein that maintains biological activity.

A proteolysis targeting chimera (PROTAC) refers to a ubiquitin pathway protein binding moiety (e.g., for an E3 ubiquitin ligase, alone or in complex with an E2 ubiquitin conjugating enzyme which is responsible for the transfer of ubiquitin to targeted proteins) and a protein targeting moiety which are linked or coupled together, wherein the ubiquitin pathway protein binding moiety recognizes a ubiquitin pathway protein and the targeting moiety recognizes a target protein (e.g., EGFR). Such compounds may be referred to herein as PROTAC compounds or PROTACs. Non-limiting examples of PROTACs include those described e.g., in ACS Med Chem Lett 10, 1549 (2019), ACS Med Chem Lett 13, 278 (2022), Bioorg Mol Chem Lett 30, 127167 (2020), Cancers 11, 1094 (2019), Chem Comm 57, 12852 (2021), Drug Dev Res 82, 422 (2020), Eur J Med Chem 189, 112061 (2020), Eur J Med Chem 192, 112199 (2020), Eur J Med Chem 218 113328 (2021), Eur J Org Chem 208, 112781 (2020), J Med Chem 65, 4709 (2022), JMC 65, 5057 (2022), Nature Chem Biol 16, 577(2020), Sig Trans Target Ther 5, 214 (2020), WO 2019/121562, and WO 2021/127561, each of which is incorporated herein by reference. For example, the PROTAC can be

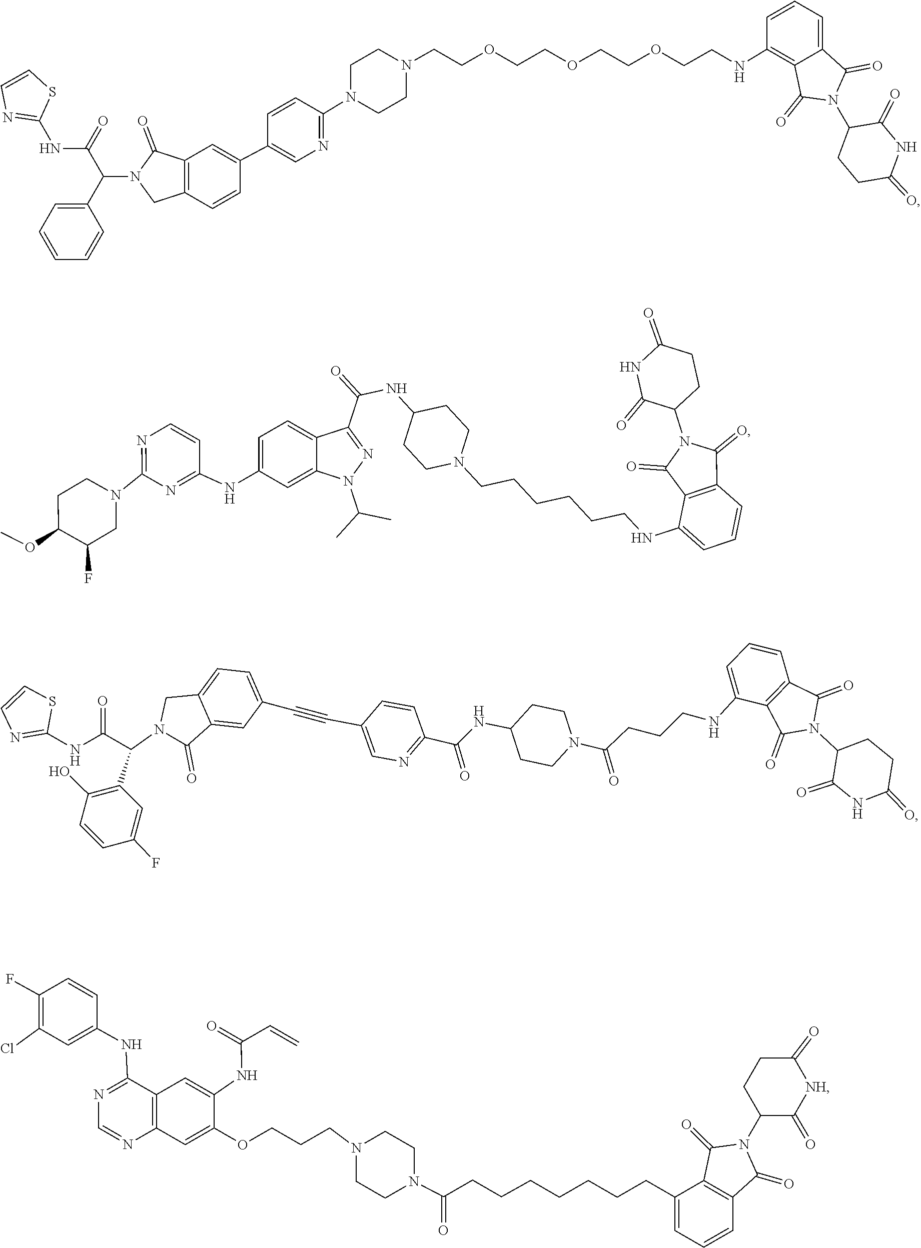 [SMILES: O=C1CCC(N2C(=O)c3cccc(NCCOCCOCCOCCN4CCN(c5ccc(-c6ccc7c(c6)C(=O)N(C(C(=O)Nc6nccs6)c6ccccc6)C7)cn5)CC4)c3C2=O)C(=O)N1 ; CO[C@H]1CCN(c2nccc(Nc3ccc4c(C(=O)NC5CCN(CCCCCCNc6cccc7c6C(=O)N(C6CCC(=O)NC6=O)C7=O)CC5)nn(C(C)C)c4c3)n2)C[C@H]1F ; O=C1CCC(N2C(=O)c3cccc(NCCCC(=O)N4CCC(NC(=O)c5ccc(C#Cc6ccc7c(c6)C(=O)N([C@@H](C(=O)Nc6nccs6)c6cc(F)ccc6O)C7)cn5)CC4)c3C2=O)C(=O)N1 ; C=CC(=O)Nc1cc2c(Nc3ccc(F)c(Cl)c3)ncnc2cc1OCCCN1CCN(C(=O)CCCCCCCc2cccc3c2C(=O)N(C2CCC(=O)NC2=O)C3=O)CC1]

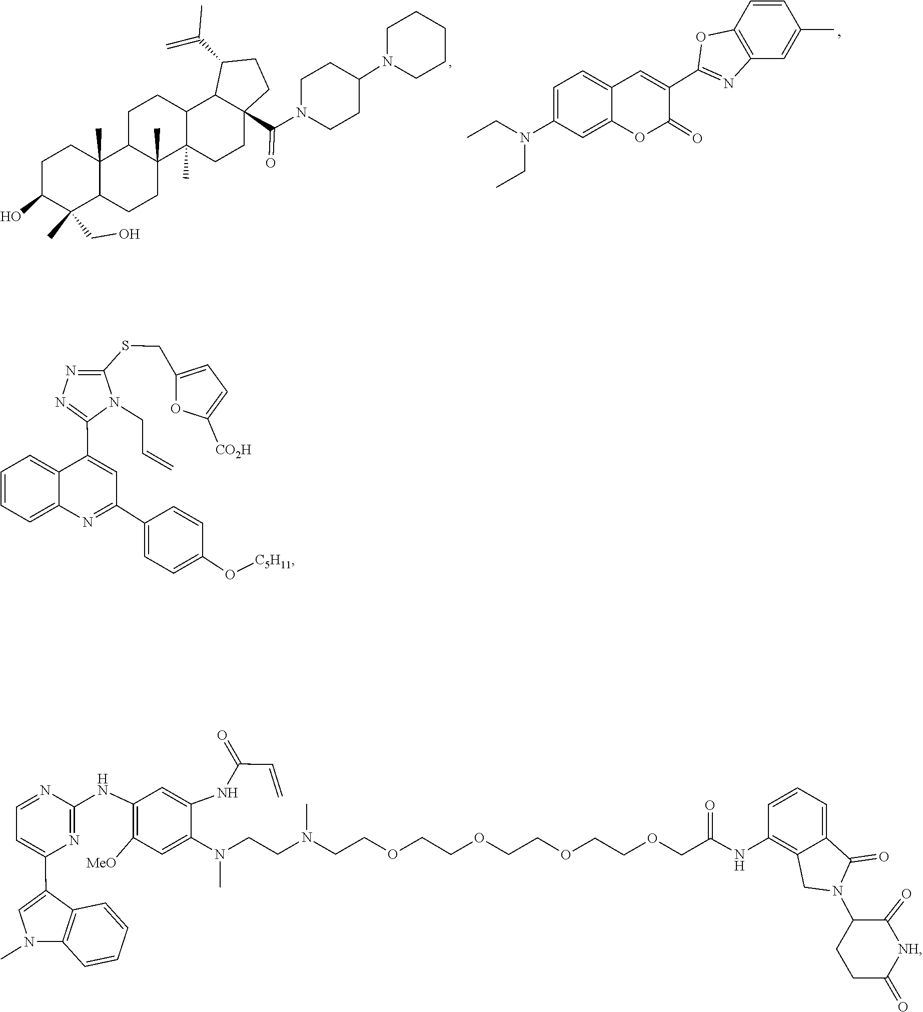

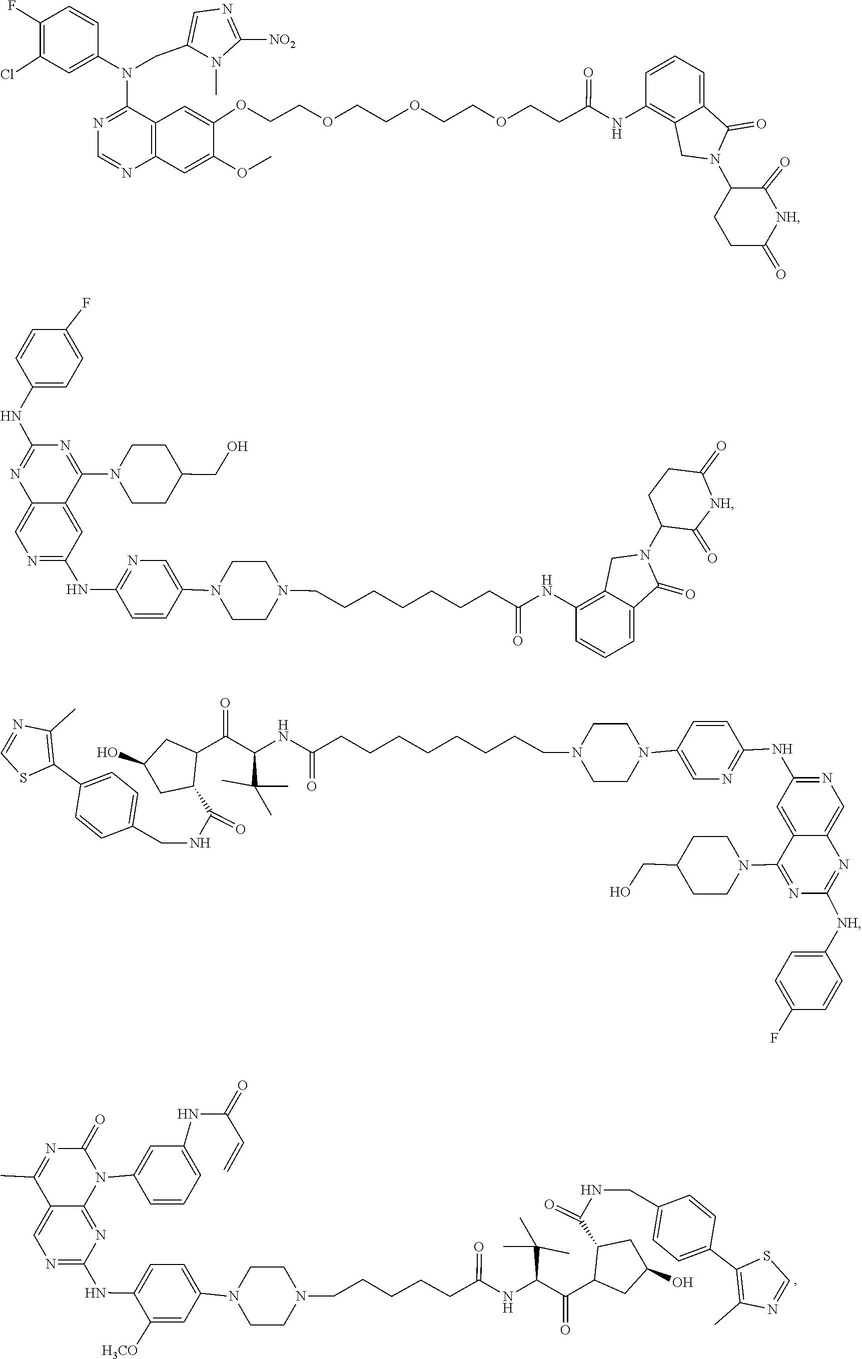

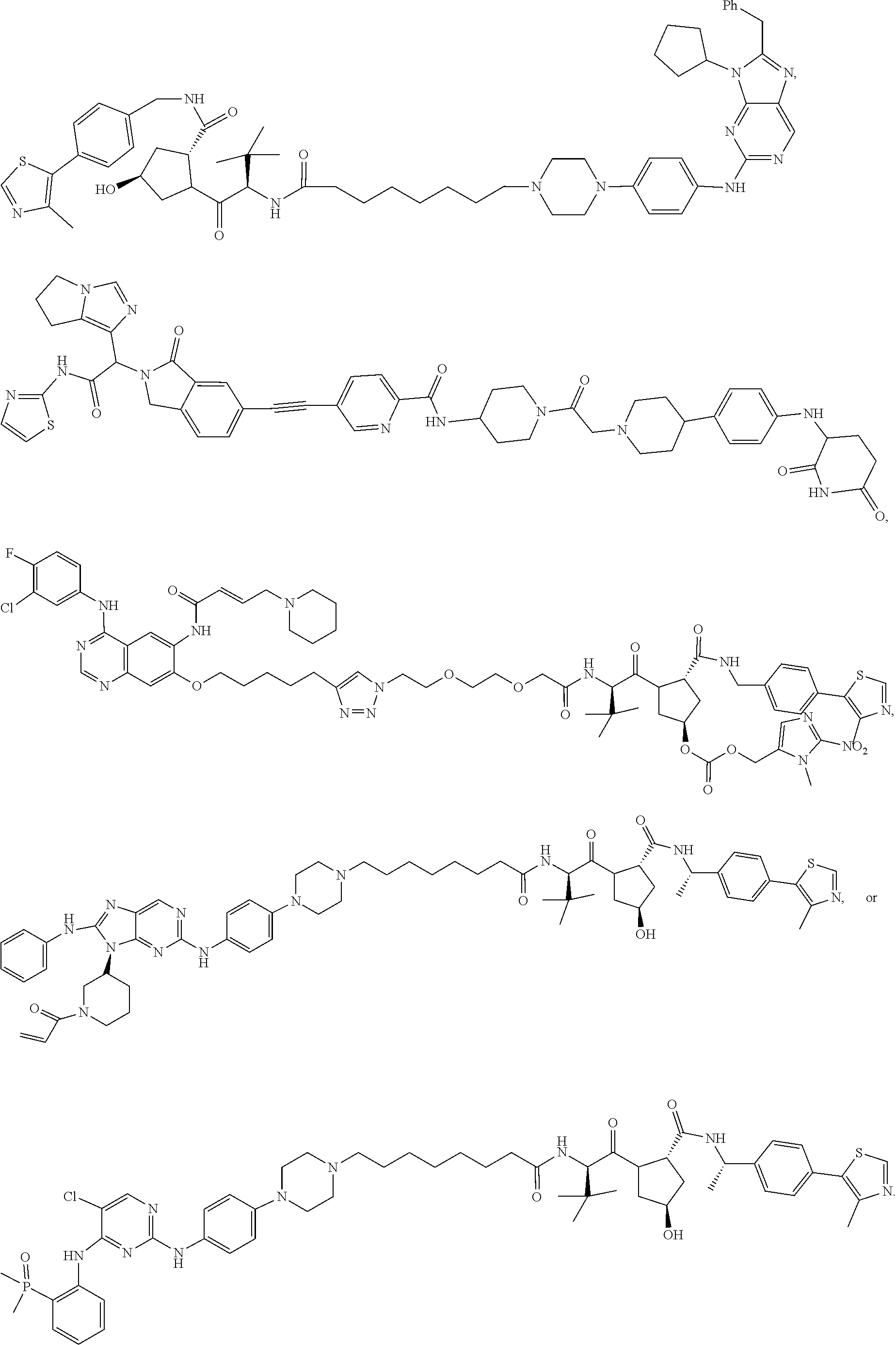

As used herein, the term “nucleic acid molecule,” “nucleotide,” “oligonucleotide,” “polynucleotide,” and “nucleic acid” are used interchangeably herein to refer to polymeric forms of nucleotides of any length. They can include both double- and single-stranded sequences and include, but are not limited to, cDNA from viral, prokaryotic, and eukaryotic sources; mRNA; genomic DNA sequences from viral (e.g., DNA viruses and retroviruses) or prokaryotic sources; RNAi; cRNA; antisense molecules; ribozymes; and synthetic DNA sequences. The term also captures sequences that include any of the known base analogs of DNA and RNA.

An aptamer, as used herein, refers to oligonucleotide or peptide sequences with the capacity to recognize a target molecule with high affinity and specificity. While aptamers can exist naturally, they are typically prepared by screening a large random sequence pool for affinity and specificity for the desired target.

The term “peptidomimetic” generally refers to a peptide, partial peptide or non-peptide molecule that mimics the tertiary binding structure or activity of a selected native peptide or protein functional domain (e.g., binding motif or active site). These peptidomimetics include recombinantly or chemically modified peptides, as well as non-peptide agents such as small molecule drug mimetics.

In various embodiments, the EGFR degrader can be a compound or salt thereof having a structure of Formula I:

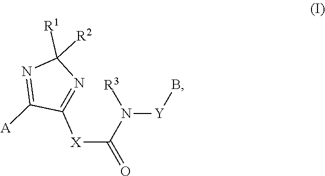

wherein X is C₁₋₆ alkylene, C₂₋₆ alkenylene, C₂₋₆ alkynylene, C₃₋₁₀ cycloalkylene, 4-6 membered heterocycle, O—C₀₋₆alkylene, O—C₂₋₆ alkenylene, O—C₂₋₆ alkynylene, O—C₃₋₁₀ cycloalkylene, O-(4-6 membered heterocyclene), S—C₀₋₆alkylene, S—C₂₋₆ alkenylene, S—C₂₋₆ alkynylene, S—C₃₋₁₀cycloalkylene, S-(4-6 membered heterocyclene), NR³—C₀₋₆alkylene, NR³—C₂₋₆ alkenylene, NR³—C₂₋₆ alkynylene, NR³—C₃₋₁₀ cycloalkylene, or NR³—(4-6 membered heterocyclene), and X is optionally substituted with 1-5 groups independently selected from R³; Y is C₀₋₆alkylene, C₃₋₆alkenylene, or C₃₋₆alkynylene, and Y is optionally substituted with 1-3 groups independently selected from halo, N(R³)₂, and R³; A is C₆₋₁₀ aryl or 5-10 membered heteroaryl having 1-4 heteroatoms selected from N, O, and S, and A is optionally substituted with 1 to 3 R⁴; B is C₆0.10 aryl, 5-10 membered heteroaryl having 1-4 heteroatoms selected from N, O, and S, 3-8 membered cycloalkyl ring, or a 4-10 membered heterocycle having 1-3 heteroatoms selected from N, O, and S, and B is optionally substituted with 1 to 3R⁵; R¹ and R² are each independently C₁₋₆ alkyl, C₃₋₆ alkenyl, C₃₋₆ alkynyl, or C₃₋₆ cycloalkyl, or R¹ and R² together with the carbon atom to which they are attached form a 4-8 membered cycloalkyl or heterocycle, wherein the heterocycle has 1 or 2 ring heteroatoms selected from O, S, and N, and wherein said cycloalkyl or heterocycle is optionally substituted with 1-2 R⁴; each R³ is independently OH, C₁₋₆ alkyl, C₂₋₆alkenyl, C₂₋₆alkynyl, C₁₋₆alkoxy, phenyl, O-phenyl, benzyl, O-benzyl, C₃₋₆cycloalkyl, 4-10 membered heterocycle having 1 to 4 heteroatoms selected from N, O, and S, or (O)₀₋₁-5-10 membered heteroaryl having 1 to 3 heteroatoms selected from N, O, and S, or two R³ taken together with the atom(s) to which they are attached form a C₃₋₆ cycloalkyl (e.g., C₄₋₆ cycloalkenyl), or 4-6 membered heterocycle having one heteroatom selected from N, O and S; each R⁴ and R⁵ is independently halo, NO₂, oxo, cyano, C₁₋₄ alkyl, C₁₋₄haloalkyl (e.g., CF₃, CHF₂), C₁₋₄alkoxy, C₁₋₄haloalkoxy (e.g., OCF₃, OCHF₂), C₁-4thioalkoxy, C₂₋₄alkenyl, C₂₋₄alkynyl, CHO, C(═O)R⁶, C(═O)N(R⁶)₂, S(O)₀-2R⁶, SO₂N(R⁶)₂, NH₂, NHR⁶, N(R⁶)₂, NR⁷COR⁶, NR⁷SO₂R⁶, P(═O)(R⁶)₂, C₃₋₆cycloalkyl, 4-10 membered heterocycle having 1 to 4 heteroatoms selected from N, O, and S (e.g., oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazinyl, dioxepanyl, dioxepanyloxy, dioxepanylamino, oxazepanyl, oxazepanyloxy, oxazepanylamino, diazepanyl, diazepanyloxy, or diazepanylamino); each R⁶ is independently H, C₁₋₆ alkyl, C₁₋₆ haloalkyl, C₃₋₆ alkenyl, C₃₋₆alkynyl, COOR⁷, CON(R⁷)₂, C₀₋₃alkylene-C₃₋₈cycloalkyl, C₀₋₃alkylene-C₆₋₁₀aryl, C₀₋₃alkylene-(4-10 membered heterocycle having 1-4 heteroatoms selected from N, O, and S), or C₀₋₃alkylene-(5-10 membered heteroaryl having 1-4 heteroatoms selected from N, O, and S), wherein the aryl, heterocyle, or heteroaryl is optionally substituted with 1 to 3 R⁷; and each R⁷ is independently H, C₁₋₆ alkyl, C₁₋₆ haloalkyl, C₃₋₆ alkenyl, C₃₋₆ alkynyl, C₁₋₄alkoxy, or C₁₋₄haloalkoxy.

In various embodiments, R¹ and R² are each independently C₁₋₆ alkyl. In some embodiments, R¹ and R² are each methyl.

In various embodiments, R¹ and R² together with the carbon atom to which they are attached form a 4-8 membered cycloalkyl or heterocycle. In some embodiments, R¹ and R² together with the carbon atom to which they are attached form a 5 or 6 membered cycloalkyl or heterocycle. In some embodiments, R¹ and R² together with the carbon atom to which they are attached form a cyclohexyl ring.

In various embodiments, R¹ and R² together with the carbon atom to which they are attached form a heterocycle having the structure:

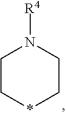

where * indicates the point of attachment to the rest of the compound of Formula I. In some embodiments, R⁴ is C₁₋₆ alkyl, C₁₋₆ haloalkyl, (C═O)R³, (C═O)OR³, CON(R³)₂, C₀₋₃alkylene-C₃₋₈cycloalkyl, C₀₋₃alkylene-C₆₋₁₀aryl, or C₀₋₃alkylene-(5-10 membered heteroaryl having 1-4 heteroatoms selected from N, O, and S), wherein the aryl or heteroaryl is optionally substituted with 1 to 3 R⁵. In some embodiments, R⁴ is C₁₋₆ alkyl, (C═O)R³, (C═O)OR³, or CON(R³)₂. In some embodiments, R⁴ is C₁₋₆ alkyl. In some embodiments, R⁴ is methyl, ethyl, propyl, isopropyl, isobutyl, or isopentyl. In some embodiments, R⁴ is methyl. In some embodiments, R⁴ is deuterated. In some embodiments, R⁴ is C₁₋₆ haloalkyl. In some embodiments, R⁴ is 3,3,3-trifluoropropyl. In some embodiments, R⁴ is C₀₋₃alkylene-C₃₋₈cycloalkyl. In some embodiments, R⁴ is cyclobutyl, cyclopentyl, or cyclohexyl. In some embodiments, R⁴ is cyclobutyl or cyclopentyl. In some embodiments, R⁴ is C₀₋₃alkylene-C₆₋₁₀aryl. In some embodiments, R⁴ is benzyl. In some embodiments, R⁴ is C₀₋₃alkylene-(5-10 membered heteroaryl having 1-4 heteroatoms selected from N, O, and S), wherein the heteroaryl is optionally substituted with 1 to 3 R⁵. In some embodiments, R⁴ is C₁alkylene-(5-10 membered heteroaryl having 1-4 heteroatoms selected from N, O, and S), wherein the heteroaryl is optionally substituted with 1 to 3 R⁵. In some embodiments, R⁴ is C₀₋₃alkylene-(5-10 membered heteroaryl having 1-4 heteroatoms selected from N, O, and S), wherein the heteroaryl is substituted with 1 to 3 R⁵. In some embodiments, R⁴ is C₀₋₃alkylene-(5-10 membered heteroaryl having 1-4 heteroatoms selected from N O and S), wherein the heteroaryl is unsubstituted. In some embodiments, R⁴ is

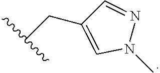 [SMILES: *Cc1cnn(C)c1]

In various embodiments, A is C₆₋₁₀ aryl. In some embodiments, A is phenyl.

In various embodiments, B is C₆₋₁₀ aryl. In some embodiments, B is phenyl. In various embodiments, B is 5-10 membered heteroaryl having 1-4 heteroatoms selected from N, O, and S. In some embodiments, B is pyridinyl. In some embodiments, B is quinolinyl. In various embodiments, B is 3-8 membered cycloalkyl. In some embodiments, B is 5 or 6 membered cycloalkyl.

In some embodiments, A is substituted with one R⁴. In some embodiments, A has the structure:

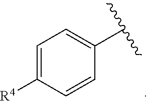

In some embodiments, A is substituted with two R⁴. In some embodiments, at least one R⁴ is C₁₋₆ alkyl. In some embodiments, at least one R⁴ is methyl. In some embodiments, at least one R⁴ is halo. In some embodiments, R⁴ is bromo. In some embodiments, at least one R⁴ is C₁₋₆ alkoxy. In some embodiments, at least one R⁴ is methoxy.

In some embodiments, B is substituted with one R⁵. In some embodiments, B is substituted with two R⁵. In some embodiments, B has the structure

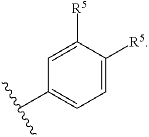

In some embodiments, at least one R⁵ is halo. In some embodiments, at least one R⁵ is fluoro or chloro. In some embodiments, one R⁵ is fluoro and the other R⁵ is chloro. In some embodiments, at least one R⁵ is C₁₋₆ alkoxy. In some embodiments, at least one R⁵ is methoxy. In some embodiments, one R⁵ is halo and the other R⁵ is C₁₋₆ alkoxy. In some embodiments, one R⁵ is chloro and the other R⁵ is methoxy.

In some embodiments, each R⁴ and R⁵ is independently C₁₋₆ alkyl, halo, or C₁₋₆ alkoxy. In some embodiments, R⁶ is C₁₋₆ alkyl, (C═O)R³, (C═O)OR³, or CON(R³)₂.

In various embodiments, X is O— C₀₋₆alkylene or S—C₀₋₆alkylene. In some embodiments, X is S—C₀₋₆alkylene. In some embodiments, X is O, S, O—CH₂—, or S—CH₂—. In various embodiments, Y is C₀₋₂alkylene. In some embodiments, Y is null or CH₂. In some embodiments, X is NR³—CH₂, O—CH₂—, or S—CH₂—, and Y is null. In some embodiments, X is NR³—CH₂, O—CH₂—, or S—CH₂—, and Y is CH₂. In some embodiments, R³ is H.

In various embodiments, X is C₁₋₆alkylene. In some embodiments, X is C₂₋₆alkenylene or C₂₋₆alkynylene. In various embodiments, Y is C₀₋₂alkylene. In some embodiments, Y is null (a bond) or CH₂. In various embodiments, Y is C₃₋₆ alkenylene or C₃₋₆ alkynylene.

As used herein, reference to an element, whether by description or chemical structure, encompasses all isotopes of that element unless otherwise described. By way of example, the term “hydrogen” or “H” in a chemical structure as used herein is understood to encompass, for example, not only ¹H, but also deuterium (²H), tritium (³H), and mixtures thereof unless otherwise denoted by use of a specific isotope. Other specific non-limiting examples of elements for which isotopes are encompassed include carbon, phosphorous, idodine, and fluorine.

It is understood that, in any compound disclosed herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. Further, compounds provided herein may be scalemic mixtures. Moreover, in any compound disclosed herein having more than one chiral center, then all diastereomers of that compound are embraced. In addition, it is understood that in any compound having one or more double bond(s) generating geometrical isomers that can be defined as E or Z each double bond may independently be E or Z or a mixture thereof. Likewise, all tautomeric forms are also intended to be included.

In some cases, the EGFR degrader of the disclosed methods comprises Compound A or a pharmaceutically acceptable salt thereof:

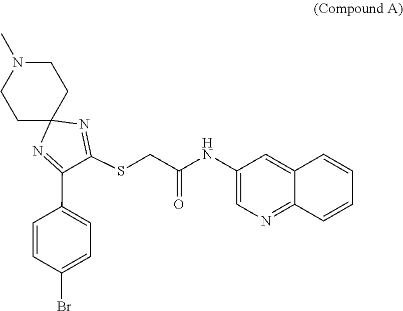 [SMILES: CN1CCC2(CC1)N=C(SCC(=O)Nc1cnc3ccccc3c1)C(c1ccc(Br)cc1)=N2]

### Chemical Definitions

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₇ alkyl refers to an alkyl group having a number of carbon atoms encompassing the entire range (e.g., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. Unless otherwise indicated, an alkylene group can be an unsubstituted alkylene group or a substituted alkylene group. “Alkenylene” and “alkynylene” are similarly defined, but for alkene or alkyne groups.

As used herein, the term “cycloalkyl” refers to a cyclic hydrocarbon group containing three to eight carbon atoms (e.g., 3, 4, 5, 6, 7, or 8 carbon atoms). The term Cₙmeans the cycloalkyl group has “n” carbon atoms. For example, C₅ cycloalkyl refers to a cycloalkyl group that has 5 carbon atoms in the ring. C₆-C₈ cycloalkyl refers to cycloalkyl groups having a number of carbon atoms encompassing the entire range (e.g., 6 to 8 carbon atoms), as well as all subgroups (e.g., 6-7, 7-8, 6, 7, and 8 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycle group, an aryl group and/or a heteroaryl group. When a cycloalkyl group is fused to another cycloalkyl group, then each of the cycloalkyl groups can contain three to eight carbon atoms unless specified otherwise. Unless otherwise indicated, a cycloalkyl group can be unsubstituted or substituted.

As used herein, the term “heterocycle” is defined similarly as cycloalkyl, except the ring contains one to three heteroatoms independently selected from oxygen, nitrogen, and sulfur. In particular, the term “heterocycle” refers to a monocyclic ring or fused bicyclic ring containing a total of three to twelve atoms (e.g., 3-8, 5-8, 3-6, 3, 4, 5, 6, 7, 8, 9, 10, 11, or 12), of which 1, 2, or 3 of the ring atoms are heteroatoms independently selected from the group consisting of oxygen, nitrogen, and sulfur, and the remaining atoms in the ring are carbon atoms. Nonlimiting examples of heterocycle groups include piperdine, pyrazolidine, tetrahydrofuran, tetrahydropyran, dihydrofuran, morpholine, and the like. The heterocycle groups described herein can be isolated or fused to a cycloalkyl group, an aryl group, and/or a heteroaryl group. Unless otherwise indicated, a heterocycle group can be unsubstituted or substituted.

Cycloalkyl and heterocycle groups are non-aromatic but can be partially unsaturated ring; and can be optionally substituted with, for example, one to five or one to three groups, independently selected alkyl, alkyleneOH, C(O)NH₂, NH₂, oxo (═O), aryl, alkylenehalo, halo, and OH. Heterocycle groups optionally can be further N-substituted with alkyl (e.g., methyl or ethyl), alkylene-OH, alkylenearyl, and alkyleneheteroaryl. Other substitutions for specific heterocycles and cycloalkyl groups are described herein.

As used herein, the term “aryl” refers to a monocyclic or bicyclic aromatic group, having 6 to 10 ring atoms. Unless otherwise indicated, an aryl group can be unsubstituted or substituted with one or more, and in particular one to five, or one to four or one to three, groups independently selected from, for example, halo, alkyl, alkenyl, OCF₃, NO₂, CN, NC, OH, alkoxy, amino, CO₂H, CO₂alkyl, aryl, and heteroaryl. Aryl groups can be isolated (e.g., phenyl) or fused to a cycloalkyl group (e.g. tetraydronaphthyl), a heterocycle group, and/or a heteroaryl group.

As used herein, the term “heteroaryl” refers to a monocyclic or bicyclic aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl group is substituted with one or more of alkyl and alkoxy groups. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “alkoxy” or “alkoxyl” as used herein refers to a “—O-alkyl” group. The alkoxy or alkoxyl group can be unsubstituted or substituted.

As used herein, “halo” refers to F, C₁, I, or Br.

As used herein, the term “therapeutically effective amount” means an amount of a compound or combination of therapeutically active compounds that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans).

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein the terms “treating”, “treat” or “treatment” and the like include preventative (e.g., prophylactic) and palliative treatment.

As used herein, the term “excipient” means any pharmaceutically acceptable additive, carrier, diluent, adjuvant, or other ingredient, other than the active pharmaceutical ingredient (API).

### Pharmaceutical Compositions

The EGFR degraders disclosed herein can be formulated in a pharmaceutical composition for administration to the patient. Pharmaceutical compositions include an appropriate amount of the EGFR degrader in combination with an appropriate carrier and optionally other useful ingredients. For example, the other useful ingredients include, but not limited to, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

The pharmaceutical compositions are administered to a patient in need thereof by any route which makes the compound bioavailable. In one embodiment, the composition is a solid formulation adapted for oral administration. In another embodiment, the composition is a tablet, powder, or capsule; or the composition is a tablet. In embodiments, the composition is a liquid formulation adapted for oral administration. In embodiments, the composition is a liquid formulation adapted for parenteral administration. In embodiments, the composition is a solution, suspension, or emulsion; or the composition is a solution. In embodiments, solid form compositions can be converted, shortly before use, to liquid form compositions for either oral or parenteral administration. These particular solid form compositions are provided in unit dose form and as such are used to provide a single liquid dosage unit. These and other pharmaceutical compositions and processes for preparing the same are well known in the art. (See, for example, Remington: The Science and Practice of Pharmacy (D. B. Troy, Editor, 21st Edition, Lippincott, Williams & Wilkins, 2006).

The dosages may be varied depending on the requirement of the subject, the severity of the condition being treated and the particular agent being employed. Determination of the proper dosage for a particular situation can be determined by one skilled in the medical arts. The total daily dosage may be divided and administered in portions throughout the day or by means providing continuous delivery.

The EGFR degraders and compositions described herein may be administered initially in a suitable dosage that may be adjusted as required, depending on the desired clinical response. In certain embodiments, the EGFR degraders are administered to a subject at a daily dosage of between 0.01 to about 50 mg/kg of body weight. In other embodiments, the dose is from 1 to 1000 mg/day. In certain embodiments, the daily dose is from 1 to 750 mg/day; or from 10 to 500 mg/day.

In embodiments, the pharmaceutical composition is in unit dosage form. The composition can be subdivided into unit doses containing appropriate quantities of the EGFR degrader(s). The unit dosage form can be a tablet, capsule, or powder in a vial or ampule, or it may be the appropriate number of any of these in a packaged form. The unit dosage form can be a packaged form, the package containing discrete quantities of composition such as packeted tablets, capsules, or powders in vials or ampules. The quantity of EGFR degrader(s) in a unit dose of the composition may be varied or adjusted from about 1 mg to about 100 mg, or from about 1 mg to about 50 mg, or from about 1 mg to about 25 mg, according to the particular application.

### Cancers for Treatment

The methods disclosed herein are useful in the treatment of cancers driven by EGFR mutation, overexpression, or ligand overexpression, mutant KRAS, or mutant BRAF. In some cases, the cancer is characterized by presence of at least one deleterious KRAS mutation. A deleterious KRAS mutation can include, but is not limited to, one of the following mutations: G12D, G12C, G12V, and G13D. In various cases, the cancer may be characterized by the presence of one or more of the following EGFR mutations: L858R, T790M, C797S, S7681, del Exon 19, or a combination thereof. In various cases, the cancer may be characterized by a deleterious BRAF mutation (e.g., V600E). In various cases, the cancer is a solid tumor.

The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, leukemia (e.g., chronic lymphocytic leukemia), chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In particular aspects, the cancer is an osimertinib-resistant cancer. In some cases, the cancer is pancreatic cancer, head and neck cancer, melanoma, colon cancer, renal cancer, leukemia, or breast cancer. In some cases, the cancer is melanoma, colon cancer, renal cancer, leukemia, or breast cancer. In some cases, the cancer to be treated in a method as disclosed herein can be pancreatic cancer, colorectal cancer, head and neck cancer, lung cancer, e.g., non-small cell lung cancer (NSCLC), ovarian cancer, cervical cancer, gastric cancer, breast cancer, hepatocellular carcinoma, glioblastoma, liver cancer, malignant mesothelioma, melanoma, multiple myeloma, prostate cancer, or renal cancer. In some embodiments, the cancer is pancreatic cancer, colorectal cancer, head and neck cancer, or lung cancer. In some embodiments, the cancer is cetuximab-resistant cancer or osimertinib-resistant cancer.

### Routes of Administration of Radiation Therapy

Radiation therapy involves the use of high-energy radiation (e.g., x-rays, gamma rays, or charged particles) to damage and/or kill cancer cells and to shrink tumors. In the methods of the invention, radiation may be delivered to the patient by a machine positioned outside the body (external-beam radiation therapy), by radioactive material placed in the body near cancer (internal radiation therapy, also called brachytherapy), or by radioactive substances administered systemically (e.g., radioactive iodine) that travel through the bloodstream to the cancer. Alternatively, these delivery methods can be used in combination.

Radiation therapies which are suitable for use in the combination treatments described herein, include the use of a) external beam radiation; and b) a radiopharmaceutical agent which comprises a radiation-emitting radioisotope.

External Beam Radiation: External beam radiation therapy for the treatment of cancer uses a radiation source that is external to the patient, typically either a radioisotope, such as Co, Cs, or a high energy x-ray source such as a linear accelerator. The external source produces a collimated beam directed into the patient to the tumor site. External-source radiation therapy avoids some of the problems of internal-source radiation therapy, but it irradiates a significant volume of non-tumorous or healthy tissue in the path of the radiation beam along with the tumorous tissue. The adverse effect of irradiating healthy tissue can be reduced, while maintaining a given dose of radiation in the tumorous tissue, by projecting the external radiation beam into the patient at a variety of angles with the beams converging on the cancer (e.g., tumor) site. The particular volume elements of healthy tissue along the path of the radiation beam change, reducing the total dose to healthy tissue during the entire treatment. The irradiation of healthy tissue also can be reduced by tightly collimating the radiation beam to the general cross section of the cancer (e.g. tumor) taken perpendicular to the axis of the radiation beam.

Radiopharmaceutical Agents: A “radiopharmaceutical agent” refers to a pharmaceutical agent which contains at least one radiation-emitting radioisotope. Radiopharmaceutical agents are routinely used in nuclear medicine for the diagnosis and/or therapy of various diseases. The radiolabeled pharmaceutical agent, for example, a radiolabeled antibody, contains a radioisotope (RI) which serves as the radiation source. As contemplated herein, the term “radioisotope’ includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule. A “metallic radioisotope” is any suitable metallic radioisotope useful in a therapeutic or diagnostic procedure in vivo or in vitro. Identifying the most appropriate isotope for radiotherapy requires weighing a variety of factors. These include tumor uptake and retention, blood clearance, rate of radiation delivery, half-life and specific activity of the radioisotope, and the feasibility of large-scale production of the radioisotope in an economical fashion. The key point for a therapeutic radiopharmaceutical is to deliver the requisite amount of radiation dose to the tumor cells and to achieve a cytotoxic or tumoricidal effect while not causing unmanageable side-effects. It is preferred that the physical half-life of the therapeutic radioisotope be similar to the biological half-life of the radiopharmaceutical at the tumor site. For example, if the half-life of the radioisotope is too short, much of the decay will have occurred before the radiopharmaceutical has reached maximum target/background ratio. On the other hand, too long a half-life would cause unnecessary radiation dose to normal tissues. Ideally, the radioisotope should have a long enough half-life to attain a minimum dose rate and to irradiate all the cells during the most radiation sensitive phases of the cell cycle. In addition, the half-life of a radioisotope must be long enough to allow adequate time for manufacturing, release, and transportation.

The type of radiation that is suitable for use in the methods of the present invention can vary. For example, radiation can be electromagnetic or particulate in nature. Electromagnetic radiation useful in the practice of this invention includes but is not limited to x-rays and gamma rays. Particulate radiation useful in the practice of this invention includes, but is not limited to, electron beams (beta particles), protons beams, neutron beams, alpha particles, and negative pi mesons. The radiation can be delivered using conventional radiological treatment apparatus and methods, and by intraoperative and stereotactic methods. Additional discussion regarding radiation treatments suitable for use in the practice of this invention can be found throughout Steven A. Leibel et al., Textbook of Radiation Oncology (1998) (publ. W. B. Saunders Company), e.g., in Chapters 13 and 14. Radiation can also be delivered by other methods such as targeted delivery, for example by radioactive “seeds,” or by systemic delivery of targeted radioactive conjugates. J. Padawer et al., Int. J. Radiat. Oncol. Biol. Phys. 7:347-357 (1981).

For tumor therapy, both α- and β-particle emitters have been investigated. Alpha particles are particularly good cytotoxic agents because they dissipate a large amount of energy within one or two cell diameters. The β-particle emitters have relatively long penetration range (2-12 mm in the tissue) depending on the energy level. The long-range penetration is particularly important for solid tumors that have heterogeneous blood flow and/or receptor expression. The β-particle emitters yield a more homogeneous dose distribution even when they are heterogeneously distributed within the target tissue.

Administration of External Beam Radiation: For administration of external beam radiation, the amount can be at least about 1 Gray (Gy) fractions at least once every other day to a treatment volume. In some embodiments, the radiation is administered in at least about 2 Gray (Gy) fractions at least once per day to a treatment volume. In another particular embodiment, the radiation is administered in at least about 2 Gray (Gy) fractions at least once per day to a treatment volume for five consecutive days per week. In some embodiments, radiation is administered in 10 Gy fractions every other day, three times per week to a treatment volume. In some embodiments, a total of at least about 20 Gy is administered to a patient in need thereof. In some embodiments, at least about 30 Gy is administered to a patient in need thereof. In some embodiments, at least about 40 Gy is administered to a patient in need thereof. Typically, the patient receives external beam therapy four or five times a week. An entire course of treatment usually lasts from one to seven weeks depending on the type of cancer and the goal of treatment. For example, a patient can receive a dose of 2 Gy/day over 30 days.

Administration of Radiopharmaceutical Agent: There are several methods for administration of a radiopharmaceutical agent. For example, the radiopharmaceutical agent can be administered by targeted delivery or by systemic delivery of targeted radioactive conjugates, such as a radiolabeled antibody, a radiolabeled peptide and a Liposome delivery System. In some embodiments, the radiolabeled pharmaceutical agent can be a radiolabeled antibody. See, for example, Ballangrud A. M., et al. Cancer Res., 2001; 61:2008-2014 and Goldenberg, D M J. Nucl. Med., 2002; 43(5):693-713, the contents of which are incorporated by reference herein. In some embodiments, the radiopharmaceutical agent can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. See, for example, Emfietzoglou D, Kostarelos K, SgouroS G. An analytical dosimetry Study for the use of radionuclide-liposome conjugates in internal radiotherapy. J Nucl Med 2001; 42:499-504, the contents of which are incorporated by reference herein. In some embodiments, the radiolabeled pharmaceutical agent can be a radiolabeled peptide. See, for example, Weiner R E, Thakur M L. Radiolabeled peptides in the diagnosis and therapy of oncological diseases. Appl Radiat Isot 2002 November; 57(5):749 63, the contents of which are incorporated by reference herein.

In addition to targeted delivery, Brachytherapy can be used to deliver the radiopharmaceutical agent to the target site. Brachytherapy is a technique that puts the radiation sources as close as possible to the tumor site. Often the source is inserted directly into the tumor. The radioactive sources can be in the form of wires, seeds or rods. Generally, cesium, iridium or iodine are used. There a two types of brachytherapy: intercavitary treatment and interstitial treatment. In intracavitary treatment, containers that hold radioactive sources are put in or near the tumor. The sources are put into the body cavities. In interstitial treatment the radioactive sources alone are put into the tumor. These radioactive sources can stay in the patient permanently. Most often, the radioactive sources are removed from the patient after several days. The amount of radiation necessary can be determined by one of skill in the art based on known doses for a particular type of cancer. See, for example, Cancer Medicine 5″ ed., Edited by R. C. Bast et al., July 2000, B C Decker, the entire content of which is hereby incorporated by reference.

In view of the many possible embodiments to which the principles of the disclosure may be applied, it should be recognized that the illustrated embodiments are only examples and should not be taken as limiting the scope of the invention.

### Examples

Mice bearing locally advanced xenograft (PC9, RKO, or UMSCC74B) were treated with vehicle (control); daily oral gavage of Compound A (also referred to as DPI-503), radiation plus vehicle, or a combination of daily oral gavage of Compound A and radiation. Tumor volumes were measured using calipers at least three times a week.

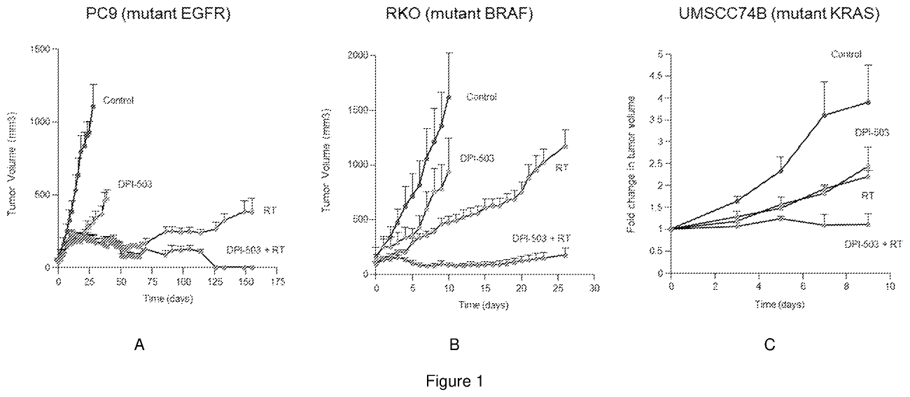

FIG. 1 A shows mice having osimertinib-resistant PC9 non-small cell lung cancer xenografts treated with vehicle, Compound A (75 mg/kg five times a week for 3 weeks), radiation (2 Gy/day five times a week for 3 weeks), or a combination of radiation and Compound A. At Day 155, when the experiment was terminated, active tumor could not be detected by gross pathology, or histochemistry.

FIG. 1B shows mice having RKO BRAF V600E colorectal cancer tumors treated with vehicle (control), Compound A (100 mg/kg daily for 8 days), radiation (2 GY/day five days a week for 3 weeks) plus vehicle, or combination of Compound A and radiation, where combination treatment mice continued Compound A administration beyond 4 weeks.

FIG. 1C shows treatment of mice having UMSCC74B KRAS G12D head and neck squamous cell carcinoma tumor with vehicle (control), Compound A (30 mg/kg biw for 2 weeks), radiation (2 GY/day, five days a week for 3 weeks) plus vehicle, or radiation and Compound A. This experiment was terminated when the control animals had to be euthanized due to tumor size.

The data shown in FIG. 1 indicate that Compound A and radiation unexpectedly exhibited efficacy in treating mutant EGFR, mutant BRAF, and mutant KRAS driven cancers, compared to either Compound A alone or radiation alone.

What is claimed:

1. A method of treating cancer in a patient suffering therefrom comprising administering to the patient an EGFR degrader, and subjecting the patient to radiation to treat the cancer.

2. The method of claim 1, wherein the EGFR degrader degrades wild type EGFR.

3. The method of claim 1 or 2, wherein the EGFR degrader degrades mutant EGFR.

4. The method of any one of claims 1-3, wherein the EGFR degrader is a compound (or pharmaceutically acceptable salt thereof), an antibody, a protein, a peptide, a PROTAC (proteolysis targeting chimera), a virus, an antibody-drug conjugate, an aptamer, a peptidomimetic agent, or an oligonucleotide.

5. The method of claim 4, wherein the compound has a structure of Formula (1) wherein 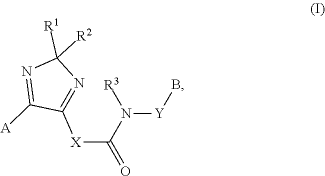
X is C₁₋₆alkylene, C₂₋₆alkenylene, C₂₋₆alkynylene, C₃₋₁₀ cycloalkylene, 4-6 membered heterocycle, O—C_{0-6 alkylene}, O—C₂₋₆ alkenylene, O—C₂₋₆ alkynylene, O—C₃₋₁₀ cycloalkylene, O-(4-6 membered heterocyclene), S—C_{0-6 alkylene}, S—C₂₋₆ alkenylene, S—C₂₋₆ alkynylene, S—C₃₋₁₀ cycloalkylene, S-(4-6 membered heterocyclene), NR³—C₀₋₆alkylene, NR³—C₂₋₆ alkenylene, NR³—C₂-s alkynylene, NR³—C₃₋₁₀ cycloalkylene, or NR³-(4-6 membered heterocyclene), and X is optionally substituted with 1-5 groups independently selected from R³;
Y is C₀₋₆alkylene, C₃₋₆alkenylene, or C₃₋₆alkynylene, and Y is optionally substituted with 1-3 groups independently selected from halo, N(R³)₂, and R³;
A is C₆₋₁₀ aryl or 5-10 membered heteroaryl having 1-4 heteroatoms selected from N, O, and S, and A is optionally substituted with 1 to 3 R⁴;
B is C₆₋₁₀ aryl, 5-10 membered heteroaryl having 1-4 heteroatoms selected from N, O, and S, 3-8 membered cycloalkyl ring, or 3-12 membered heterocycle having 1-3 ring heteroatoms selected from O, S, and N, and B is optionally substituted with 1 to 3 R⁵;
R¹ and R² are each independently C₁₋₆ alkyl, C₃₋₆ alkenyl, C₃₋₆ alkynyl, or C₃₋₆ cycloalkyl, or R¹ and R² together with the carbon atom to which they are attached form a 4-8 membered cycloalkyl or heterocycle, wherein the heterocycle has 1 or 2 ring heteroatoms selected from O, S, and N, and wherein said cycloalkyl or heterocycle is optionally substituted with 1-2 R⁴; each R³ is independently OH, C₁₋₆ alkyl, C₁₋₆alkoxy, phenyl, O-phenyl, benzyl, O-benzyl, or (O)₀₋₁-5-10 membered heteroaryl having 1 to 3 heteroatoms selected from N, O, and S, or two R³ taken together with the atom(s) to which they are attached form a C₃₋₆ cycloalkyl (e.g., C₄₋₆ cycloalkenyl), or 4-6 membered heterocycle having one heteroatom selected from N, O and S;
each R⁴ and R⁵ is independently halo, NO₂, oxo, cyano, C₁₋₄ alkyl, C₁₋₄haloalkyl, C₁₋₄alkoxy, C₁₋₄haloalkoxy, C₁0.4thioalkoxy, CHO, C(═O)R⁶, C(═O)N(R⁶)₂, S(O)₀₋₂R⁶, SO₂N(R⁶)₂, NH₂, NHR⁶, N(R⁶)₂, NR⁷COR⁶, NR⁷SO₂R⁶, P(═O)(R⁶)₂, oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazinyl, dioxepanyl, dioxepanyloxy, dioxepanylamino, oxazepanyl, oxazepanyloxy, oxazepanylamino, diazepanyl, diazepanyloxy, or diazepanylamino;
each R⁶ is independently H, C₁₋₆ alkyl, C₁₋₆ haloalkyl, C₃₋₆ alkenyl, C₃₋₆ alkynyl, COOR⁷, CON(R⁷)₂, C₀₋₃alkylene-C₃₋₈cycloalkyl, C₀₋₃alkylene-C₆₋₁₀aryl, or C₀₋₃alkylene-(5-10 membered heteroaryl having 1-4 heteroatoms selected from N, O, and S), wherein the aryl or heteroaryl is optionally substituted with 1 to 3 R⁷; and
each R⁷ is independently H, C₁₋₆ alkyl, C₁₋₆ haloalkyl, C₃₋₆ alkenyl, C₃₋₆ alkynyl, C₁₋₄alkoxy, or C₁₋₄haloalkoxy,
or a pharmaceutically acceptable salt thereof.

6. The method of claim 5, wherein R¹ and R² are each independently C₁₋₆ alkyl.

7. The method of claim 6, wherein R¹ and R² are each methyl.

8. The method of claim 5, wherein R¹ and R² together with the carbon atom to which they are attached form a 4-8 membered cycloalkyl or heterocycle.

9. The method of claim 8, wherein R¹ and R² together with the carbon atom to which they are attached form a 5 or 6 membered cycloalkyl or heterocycle.

10. The method of claim 9, wherein R¹ and R² together with the carbon atom to which they are attached form a cyclohexyl ring.

11. The method of claim 9, wherein R¹ and R² together with the carbon atom to which they are attached form a heterocycle having the structure: 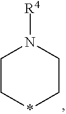
where * indicates the point of attachment to the rest of the compound of Formula I.

12. The method of any one of claims 5 to 11, wherein A is C₆₋₁₀ aryl.

13. The method of claim 12, wherein A is phenyl.

14. The method of any one of claims 5 to 13, wherein B is C₆0.10 aryl.

15. The method of claim 14, wherein B is phenyl.

16. The method of any one of claims 5 to 13, wherein B is 5-10 membered heteroaryl having 1-4 heteroatoms selected from N, O, and S.

17. The method of claim 16, wherein B is pyridinyl.

18. The method of claim 16, wherein B is quinolinyl.

19. The method of any one of claims 5 to 13, wherein B is 3-8 membered cycloalkyl.

20. The method of claim 19, wherein B is 5 or 6 membered cycloalkyl.

21. The method of any one of claims 5 to 13, wherein B is 3-12 membered heterocycle having 1-3 ring heteroatoms selected from O, S, and N.

22. The method of any one of claims 5 to 21, wherein A is substituted with one R⁴.

23. The method of claim 22, wherein A has the structure:

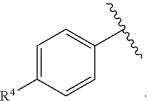

24. The method of any one of claims 5 to 21, wherein A is substituted with two R⁴.

25. The method of any one of claims 5 to 24, wherein at least one R⁴ is C₁₋₆ alkyl.

26. The method of claim 25, wherein is at least one R⁴ is methyl.

27. The method of any one of claims 5 to 26, wherein at least one R⁴ is halo.

28. The method of claim 27, wherein R⁴ is bromo.

29. The method of claim 27 or 28, wherein R⁴ is chloro.

30. The method of claim 27, 28, or 29, wherein R⁴ is fluoro.

31. The method of any one of claims 5 to 30, wherein at least one R⁴ is C₁₋₆ alkoxy.

32. The method of claim 31, wherein at least one R⁴ is methoxy.

33. The method of any one of claims 5 to 32, wherein B is substituted with one R⁵.

34. The method of any one of claims 5 to 32, wherein B is substituted with two R⁵.

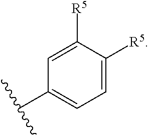

35. The method of claim 34, wherein B has the structure.

36. The method of any one of claims 5 to 35, wherein at least one R⁵ is halo.

37. The method of claim 36, wherein at least one R⁵ is fluoro or chloro.

38. The method of claim 34 or 36, wherein one R⁵ is fluoro and the other R⁵ is chloro.

39. The method of any one of claims 5 to 38, wherein at least one R⁵ is C₁₋₆alkoxy.

40. The method of claim 39, wherein at least one R⁵ is methoxy.

41. The method of any one of claims 34 to 40, wherein one R⁵ is halo and the other R⁵ is C₁₋₆alkoxy.

42. The method of claim 41, wherein one R⁵ is chloro and the other R⁵ is methoxy.

43. The method of any one of claims 5 to 42, wherein X is C₁₋₆alkylene.

44. The method of any one of claims 5 to 42, wherein X is C₂₋₆alkenylene or C₂₋₆alkynylene.

45. The method of any one of claims 5 to 42, wherein X is C₃₋₁₀ cycloalkylene, or 4-6 membered heterocyclene.

46. The method of any one of claims 5 to 42, wherein X is O— C₀₋₆alkylene or S—C₀₋₆alkylene.

47. The method of claim 46, wherein X is O, S, O—CH₂—, or S—CH₂—.

48. The method of any one of claims 5 to 47, wherein Y is a bond or CH₂.

49. The method of any one of claims 5 to 47, wherein Y is C₁₋₆alkylene.

50. The method of any one of claims 5 to 47, wherein Y is C₂₋₆alkenylene or C₂₋₆alkynylene.

51. The method of any one of claims 5 to 50, wherein R³ is H.

52. The method of claim 4, wherein the compound is Compound A or a salt thereof

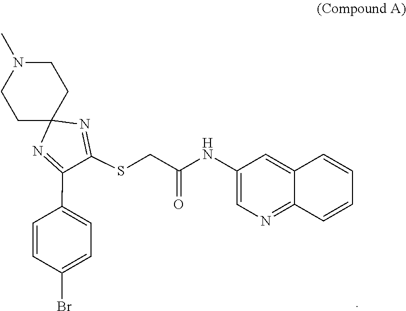 [SMILES: CN1CCC2(CC1)N=C(SCC(=O)Nc1cnc3ccccc3c1)C(c1ccc(Br)cc1)=N2]

53. The method of any one of claims 1-52, wherein the cancer is an EGFR, KRAS, or BRAF-mutated cancer.

54. The method of claim 53, wherein the KRAS mutation is G12D, G12V, G12C, or G13D, or a combination thereof.

55. The method of claim 53 or 54, wherein the KRAS mutation is G12D.

56. The method of any one of claims 53-55, wherein the EGFR mutation is L858R, T790M, C₇₉₇S, S7681, or del Exon 19, or a combination thereof.

57. The method of any one of claims 1-56, wherein the cancer is a solid tumor.

58. The method of any one of claims 1-57, wherein the cancer is pancreatic cancer, colorectal cancer, head and neck cancer, or lung cancer.

59. The method of any one of claims 1-58, wherein the EGFR degrader is administered in an amount of 1-500 mg/kg.

60. The method of claim 59, wherein the EGFR degrader is administered in an amount of 20-40 mg/kg.

61. The method of any one of claims 1-60, wherein the EGFR degrader is administered orally.

62. The method of any one of claims 1-61, wherein the radiation is administered in an amount of at least 2 Gy.
